SEQUENCE LISTING (19 sequences)

SEQ ID NO: 1 - DNA, 17 bp, Artificial
  Consensus sequence of thrombin binding aptamer
ggttggnnnnnggttgg

SEQ ID NO: 2 - DNA, 15 bp, Artificial
  Chemically synthesized oligonucleotide
ggttggtgtggttgg

SEQ ID NO: 3 - DNA, 17 bp, Artificial
  thrombin binding quadruplux region element
ggtwggnnnnnggttgg

SEQ ID NO: 4 - DNA, 44 bp, Artificial
  Chemically synthesized oligonucleotide
ctgggttggtgaggttggtcagcacggttggtgaggttggtgtg

SEQ ID NO: 5 - DNA, 48 bp, Artificial
  Chemically synthesized oligonucleotide
gctgtggttggtgaggttggcagcgcactggttggtgaggttgggtgc

SEQ ID NO: 6 - DNA, 52 bp, Artificial
  Chemically synthesized oligonucleotide
gcatgtggttggtgaggttggcatgcgctactggttggtgaggttgggtagc

SEQ ID NO: 7 - DNA, 56 bp, Artificial
  Chemically synthesized oligonucleotide
gcgatgtggttggtgaggttggcatcgcgactactggttggtgaggttgggtagtc

SEQ ID NO: 8 - DNA, 69 bp, Artificial
  Chemically synthesized oligonucleotide
gctgtggttggtgaggttggcagcagccaaggtaaccagtacaaggtgctaaacgtaatggcttcggct

SEQ ID NO: 9 - DNA, 55 bp, Artificial
  Chemically synthesized oligonucleotide
gagtccgtggtagggcaggttggggtgactcgctgtggttggtgaggttggcagc

SEQ ID NO: 10 - DNA, 56 bp, Artificial
  Chemically synthesized oligonucleotide
gagtccgtggtagggcaggttggggtgactcgctgtggttggtgaggttggacagc

SEQ ID NO: 11 - DNA, 56 bp, Artificial
  Chemically synthesized oligonucleotide
gagtccgtggtagggcaggttggggtgactcgctgcggttggtgaggttgggcagc

SEQ ID NO: 12 - DNA, 48 bp, Artificial
  Chemically synthesized oligonucleotide
gctgtggttggngaggnnggcagcgcacnggnnggngaggnngggngc

SEQ ID NO: 13 - DNA, 15 bp, Artificial
  thrombin binding region quadruplex sequence
ggtagggcaggttgg

SEQ ID NO: 14 - DNA, 27 bp, Artificial
  TCI oligonucleotide
actggttggtgaggttgggtgcgaagc

SEQ ID NO: 15 - DNA, 26 bp, Artificial
  TC1-T oligonucleotide
acggttggtgaggttgggtgcgaagc

SEQ ID NO: 16 - DNA, 28 bp, Artificial
  TC3 oligonucleotide
actggttggtgaggttgggtgcgaaagc

SEQ ID NO: 17 - DNA, 69 bp, Artificial
  TS2-1 oligonucleotide
gctgtggttggtgaggttggcagcagctggcggtacgggccgtgcacccacttacctgggaagtgagct

SEQ ID NO: 18 - DNA, 69 bp, Artificial
  TS3-1 oligonucleotide
gctgtggttggtgaggttggcagcagccattcaccatggccccttcctacgtatgttctgcgggtggct

SEQ ID NO: 19 - DNA, 48 bp, Artificial
  Chemically synthesized oligonucleotide
gcacccaacctcaccaaccagtgcgctgccaacctcaccaagcacagc

(19) United States
(12) Patent Application Publication
(10) Pub. No.: US 2005/0176940 A1
(43) Pub. Date: Aug. 11, 2005

(54) Aptamers and antiaptamers

(75) Inventor: Garry Charles King, Coogee, New South Wales (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Unisearch Limited, Kensington, New South Wales (AU)

(21) Appl. No.: 10/482,006

(22) PCT Filed: Jun. 28, 2002
(86) PCT No.: PCT/AU02/00853

(30) Foreign Application Priority Data
Jun. 29, 2001 (AU) PR 6041

(51) Int. Cl. A61K 48/00; C07H 21/02

(52) U.S. Cl. 536/23.1; 514/44

(57) ABSTRACT

The present invention relates to: An aptamer comprising a circular oligonucleotide defining one to four target binding regions; An aptamer comprising an oligonucleotide defining two, three or four thrombin binding quadruplex regions separated by at least partially duplex regions, wherein the quadruplex regions comprise a GGTMGGXGGTTGG sequence wherein M represents A or T and X represents a sequence of two to five nucleotides and/or nucleotide analogues; An aptamer represented by formula (I): 5′D₁, wQxD₁D₂yQzD₂,3′—the variables are as defined in the specification; and Aptamers selected from specific sequences.

### FIELD OF THE INVENTION

The present invention relates to aptamers, and in particular to aptamers having circular conformations and thrombin inhibitory activity. The invention also relates to compositions comprising such aptamers and methods of treatment and uses involving the aptamers, as well as to antidotes of aptamer activity.

### BACKGROUND OF THE INVENTION

The processes of blood clotting, tissue repair and clot dissolution are referred to generally as haemostasis, which requires the coordinated action of platelets, clotting factors, endothelial cells and smooth muscle cells within blood vessels (Wu, 1984). Thrombin is an essential component of the haemostatic processes and is responsible for activation of platelets to adhere to exposed subendothelial structures, conversion of soluble fibrinogen into insoluble fibrin and activation of factor XIIIa, which in turn causes crosslinking of fibrin molecules to form a hard clot.

Apart from its haemostatic functions, thrombin is recognised as having a number of other activities, for example as a mitogen (Carney et al, 1985). It is also thought to exert a chemotactic effect on monocytes (Bar Shavit and Wilner, 1986). In light of these functions thrombin has been implicated as a pro-metastatic agent (Nierodzik et al, 1992) as well as a factor involved in neurodegenerative disease (Tapparelli et al, 1993). Therefore, apart from the obvious roles of thrombin inhibitors in prevention or reduction of thrombosis and blood or blood product coagulation, thrombin inhibitors have the potential to be used in the treatment of a wide range of disorders including inflammation, cancer and neural disease.

Present anticoagulant and antithrombotic therapies rely upon the use of heparin and coumarin derivatives that indirectly and incompletely inhibit the coagulation system. The coumarins are the only class of currently available thrombin inhibitors to possess significant oral activity, which makes them acceptable to patients and useful in long term treatments. However, as a result of their mode of action which involves inhibition of hepatic synthesis of vitamin K-dependent coagulation proteins (Tapparelli et al, 1993), the coumarins are associated with a number of disadvantages. In particular the coumarins exhibit pharmacological interactions with food and other drugs, require several days for a full thrombin inhibitory effect to manifest and several days for resynthesis of coagulation factors to normalise on cessation of treatment. Coumarin therapy is also characterised by variability between patients, which necessitates close monitoring.

The most important drugs presently used in the prevention and treatment of thromboembolic diseases are the heparins, which are administered in surgery and to patients suffering from stroke, acute myocardial infarction, respiratory failure and during immobilisation of patients when extracorporeal circulation or renal dialysis is required (Stubbs and Bode, 1995). Unlike the coumarins, which take days to manifest their effects, heparin compounds have an immediate effect on blood coagulation. However, they are also associated with a wide range of biological effects due to their binding of a variety of cells including platelets, endothelial cells, red blood corpuscles and lymphocytes (Stubbs and Bode, 1995) as well as an interaction with more than fifty enzymes (Jaques, 1980). Heparin administration can be associated with side effects including heparin-associated thrombocytopenia and osteoporosis. Although there have been advances with fractionated, more orally bioavailable heparins, conventional unfractionated heparins are characterised by low oral bioavailability which means they must be parenterally administered, such that they are restricted to short term usage. A major, further limitation relating to the heparins is their ineffectiveness in treatment of arterial thrombosis (Topoi et al, 1989).

Although a number of anticoagulant agents have been trialled in treatment of thromboembolic diseases, none so far has supplanted heparin. There is therefore a pressing need to develop anticoagulant agents, that preferably are effective in the treatment of arterial thrombosis, are orally administrable and exhibit long lasting activity in vivo, with minimal side-effects.

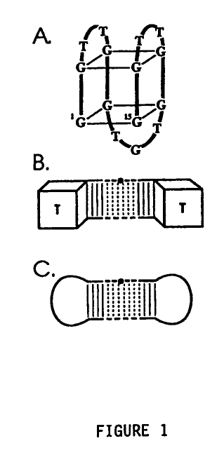

Some consideration has been given to the development of nucleic acid aptamers as antithrombotic agents. Aptamers are nucleic acids capable of three dimensional recognition that bind specific proteins or other molecules. Many known thrombin binding aptamers are composed of oligodeoxynucleotides containing the consensus sequence d(GGTTGGXGGTTGG), (<400>1), where G and T nucleotides are invariant and X is any two to five nucleotides. The 15-mer d(GGTTGGTGTGGTTGG), (<400>2), also known as GS-522 has been the subject of a number of structural and functional studies. These known thrombin-binding aptamers are characterised by a central core of two guanine quartets (Guschlbauer et al, 1990) formed from eight conserved guanine residues. These two G-quartets are linked by two TT loops at one end and a TGT loop at the other end of a quadruplex, as shown in FIG. 1A. Thrombin binding aptamers of this type have been identified as binding to thrombin exosite II (Padmanabhan et al, 1993). Although GS-522 and molecules like it have been shown to be effective in inhibiting clot- and matrix-bound thrombin (Li et al, 1994), the antithrombotic aptamers known to date have been characterised by low oral bioavailability and short in vivo half life. In an attempt to overcome problems with known thrombin binding aptamers such as those disclosed in WO 92/14842 a number of approaches have been attempted. For example, U.S. Pat. No. 5,399,676 proposes DNA-binding oligonucleotides stabilised against exonuclease degradation by virtue of combining tandem sequences of inverted polarity via a linker molecule. U.S. Pat. No. 5,668,265 discloses a bi-directional nucleic acid ligand that may be used as a diagnostic or therapeutic and which combines at least two oligonucleotides of opposite sequence polarity via a linker molecule. The publication of Macaya et al (1995) described quadruplex-duplex aptamers stabilized by either disulfide or triethylene glycol (TEG) linkages between the terminal nucleotides. Although molecules of these types demonstrate improved stability to exonuclease degradation, they are limited in their therapeutic and diagnostic utility.

It is with the difficulties associated with prior art antithrombotic agents in mind that the compounds according to the present invention have been conceived. By virtue of their molecular recognition properties, these compounds may also be employed in diagnostic applications.

### SUMMARY OF THE INVENTION

The present invention provides an aptamer comprising a circular oligonucleotide defining one to four target binding regions.

In a preferred form of the invention, the aptamer defines two, three or four target binding regions.

Preferably, the aptamer defines one or more protein, cellular, cell component or material binding regions.

A preferred cellular binding region is an L-selectin binding domain.

A preferred protein binding region is a thrombin binding region. Accordingly, in one embodiment of the present invention there is provided an aptamer comprising a circular oligonucleotide defining one to four thrombin binding regions.

Preferably, the aptamer defines two, three or four thrombin binding regions wherein said regions are separated by at least partially duplex regions. Preferably, the thrombin binding regions are quadruplex structures.

According to another embodiment of the invention there is provided an aptamer defining two, three or four thrombin binding quadruplex regions separated by at least partially duplex regions, wherein the quadruplex regions comprise a GGTMGGXGGTTGG, sequence (<400>3) wherein M represents A or T and X represents a sequence of two to five nucleotides and/or nucleotide analogues.

Preferably, the aptamer is ligated at its termini to form a circular oligonucleotide. Preferably, the termini have been enzymatically ligated, or alternatively chemically ligated.

Preferably, X represents a sequence selected from TGT, GCA and TGA.

According to another embodiment of the invention there is provided an aptamer represented by formula I:
5′ D₁′wQxD₁D₂yQzD₂′ 3′ Formula I
wherein
Q represents a sequence GGTMGGXGGTTGG where M represents A or T and X represents a sequence of two to five nucleotides and/or nucleotide analogues;
w, x, y and z are the same or different and represent a sequence of zero to ten nucleotides and/or nucleotide analogues;
D₁ and D₂ are the same or different and each represent a sequence of zero to twenty-five nucleotides and/or nucleotide analogues, with the proviso that D₁ and D₂ together comprise at least two nucleotides or nucleotide analogues;
D₁′ and D₂′ are the same or different and each represent a sequence of zero to fifty nucleotides and/or nucleotide analogues, wherein at least two consecutive nucleotides or nucleotide analogues of D₁′ and/or D₂′ are complimentary to at least two consecutive nucleotides or nucleotide analogues of D₁ and/or D₂, so as to allow duplex formation between complimentary nucleotides or nucleotide analogues.

Preferably, the 5′ terminus is phosphorylated.

Preferably, w, x, y and z are the same or different and each represent zero, one or two nucleotides and/or nucleotide analogues.

Preferably, D₁ and D₂ in total represent two to twenty nucleotides and/or nucleotide analogues. Particularly preferably, D₁ and D₂ in total represent four to twelve nucleotides and/or nucleotide analogues.

Preferably, D₁′ and D₂′ in total represent two to twenty nucleotides and/or nucleotide analogues. Particularly preferably, D₁′ and D₂′ in total represent four to twelve nucleotides and/or nucleotide analogues.

Preferably, the aptamer is ligated at its termini to form a circular oligonucleotide. Preferably, the termini have been enzymatically ligated or chemically ligated.

In a preferred embodiment of the invention the aptamer consists of nucleotides. Preferably, the aptamer consists of RNA and more preferably the aptamer consists of DNA.

Preferably, X represents a sequence selected from TGT, GCA and TGA.

Preferably, D₁ and D₁′ are selected from the following respective pairs:
CAG and CTG;
CAGC and GCTG;
CATGC and GCATG;
CATCGC and GCGATG.

Preferably, D₂ and D₂′ are selected from the following respective pairs:
CAC and GTG;
GCAC and GTGC;
GCTAC and GTAGC;
GACTAC and GTAGTC.

According to another embodiment of the invention there are provided aptamers selected from those comprising the following sequences:

| | |
|---|---|
| DH6-1 | 5′ p CTG GGT TGG TGA GGT TGG TCA GCA CGG |
| | TTG GTG AGG TTG GTG TG 3′ (<400>4) |
| DH8-1 | 5′ p GCT GTG GTT GGT GAG GTT GGC AGC GCA |
| | CTG GTT GGT GAG GTT GGG TGC 3′ (<400>5) |
| DH10-1 | 5′ p GCA TGT GGT TGG TGA GGT TGG CAT GCG |
| | CTA CTG GTT GGT GAG GTT GGG TAG C 3′ |
| | (<400>6) |
| DH12-1 | 5′ p GCG ATG TGG TTG GTG AGG TTG GCA TCG |
| | CGA CTA CTG GTT GGT GAG GTT GGG TAG TC 3′ |
| | (<400>7) |
| TS1-1 | 5′ p GCT GTG GTT GGT GAG GTT GGC AGC AGC |
| | CAA GGT AAC CAG TAC AAG GTG CTA AAC GTA |
| | ATG GCT TCG GCT 3′ (<400>8) |
| TT4-1 | 5′ GAG TCC GTG GTA GGG CAG GTT GGG GTG ACT |
| | CGC TGT GGT TGG TGA GGT TGG CAG C 3′ |
| | (<400>9) |
| TT4-2 | 5′ GAG TCC GTG GTA GGG CAG GTT GGG GTG ACT |
| | CGC TGT GGT TGG TGA GGT TGG ACA GC 3′ |
| | (<400>10) |
| TT4-3 | 5′ GAG TCC GTG GTA GGG CAG GTT GGG GTG ACT |
| | CGC TGC GGT TGG TGA GGT TGG GCA GC 3′ |
| | (<400>11) |
| DH8-Br1 | 5′ GCT GTG GTT GGB GAG GBB GGC AGC GCA CBG |
| | GBB GGB GAG GBB GGG BGC 3′ (<400>12) |

where B=5-bromo-2′-deoxyuridine, 5-iodo-2′-deoxyuridine or other photoactive nucleotide analogue.

According to another embodiment of the invention there is provided an antidote aptamer comprising at least ten nucleotides and/or nucleotide analogues complimentary to a sequence of at least ten nucleotides and/or nucleotide analogues from an aptamer as referred to above.

In one embodiment, the antidote aptamer comprises the following sequence:

| | |
|---|---|
| ADH8-1 | 5′ pGCA CCC AAC CTC ACC AAC CAG TGC GCT GCC |
| | AAC CTC ACC AAG CAC AGC 3′ (<400>19) |

In another embodiment there is provided an antisense oligonucleotide of an aptamer according to the invention.

In another embodiment there is provided a method of treatment of thrombosis in a patient requiring such treatment which comprises administering to said patient an effective amount of an aptamer according to the invention.

In another embodiment there is provided a method of preventing or reducing coagulation of blood or blood derived products which comprises contacting the blood or blood derived product with an effective amount of an aptamer according to the invention.

In another embodiment there is provided use of a compound according to the invention in preparation of a medicament for the treatment of thrombosis.

In a further embodiment, there is provided a method for capturing leukocytes from a physiological fluid comprising contacting the physiological fluid with an effective amount of an aptamer of the invention.

The invention also provides a composition comprising an aptamer of the invention together with one or more pharmaceutically acceptable carriers or excipients.

### DETAILED DESCRIPTION OF THE FIGURES

The present invention will be described further and by way of example only with reference to the following figures:

FIG. 1 Thrombin aptamer and antidote structures
(A) Canonical thrombin aptamer
(B) Schematic of divalent aptamer with G-quadruplex heads
(C) Schematic of divalent antidote aptamer

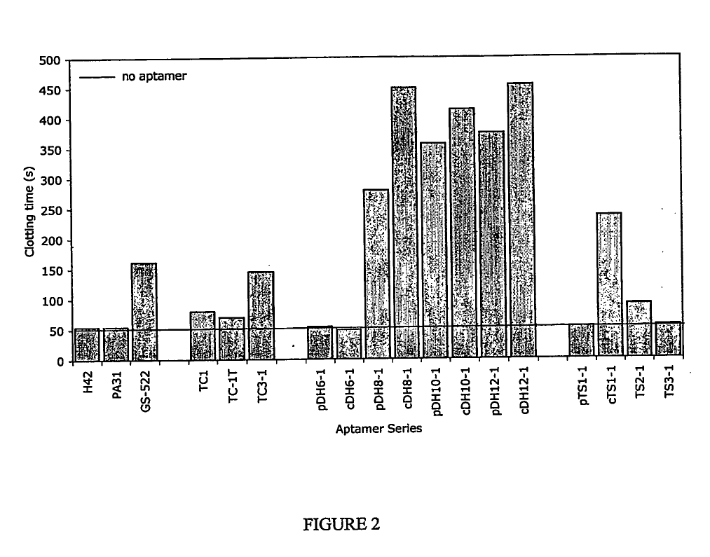

FIG. 2: Thrombin inhibition of aptamers in selection buffer

Comparative activities of TC, DH and TS aptamer families incubated at 37° C. for 1 min in selection buffer. Clotting times represent the average of at least three measurements.

Final concentrations of aptamer, thrombin and fibrinogen were 100 nM, ˜50 nM and 2 mg/mL, respectively.

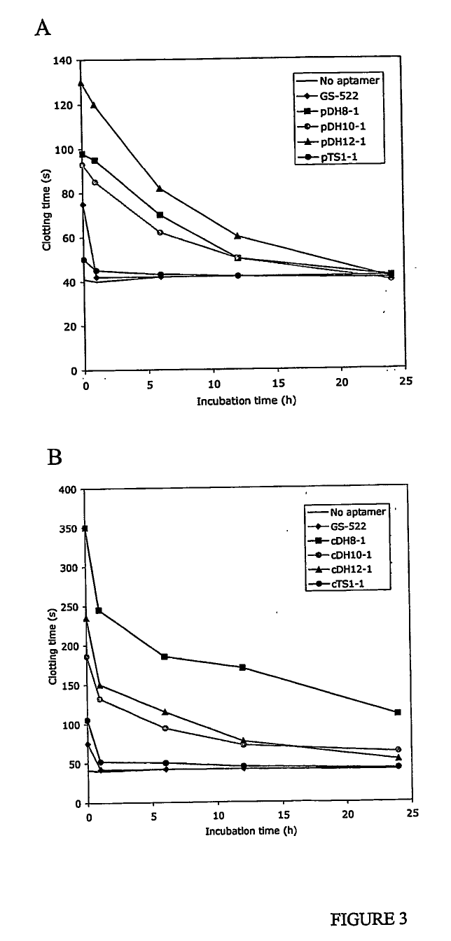

FIG. 3: Functional stability of aptamers

(A) Linear and (B) Circular aptamers incubated in 100 μL serum at 37° C. for 1 min and at 1, 6, 12, and 24 h. Clotting was initiated by the addition of thrombin and fibrinogen in selection buffer. Final concentrations: 50 nM DNA, ≈50 nM thrombin and 1.5 mg/mL fibrinogen.

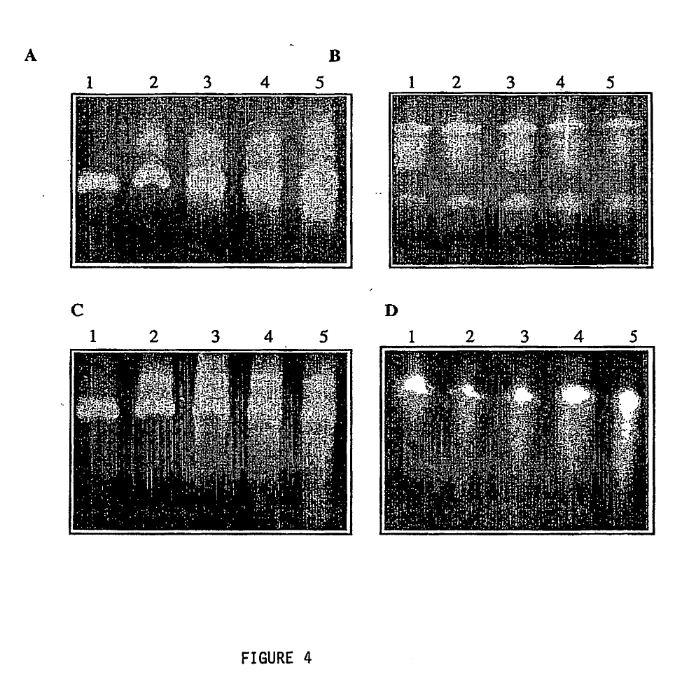

FIG. 4: Physical degradation of aptamers

Incubation of (A) cDH8-1; (B) cTS1-1; (C) cDH12-1; and (D) unligated pDH12-1 in serum at 37° C. Lanes 1-5 indicate times samples. Circular DH aptamers (A, C) were sampled at 1 min, 1, 6, 12 and 24 h. cTS1-1 (C) samples were collected at 1 min, 1, 2, 3 and 6 h. Unligated pDH12-1 (D) at 1, 15, 30, 60 and 120 min. cDH samples were run on non-denaturing PAGE; cTS1 on denaturing (urea) PAGE. Gels A, B and C were stained with SYBR II for 30 minutes before being visualised under fluorescence. Gel D was stained with ethidium bromide for UV luminescence.

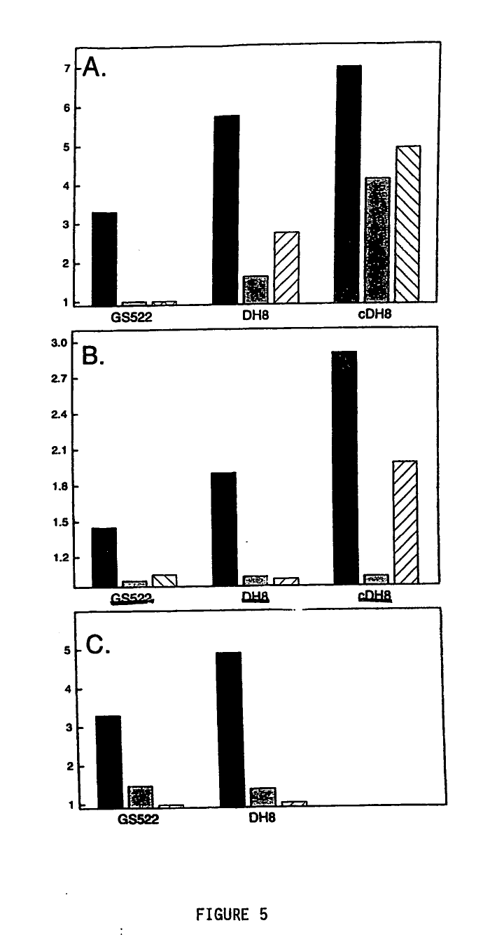

FIG. 5: Antidote activity against thrombin aptamers

Fold-anticoagulant activity (ε) for GS-522, pDH8-1 and cDH8-1 (data available for buffer and serum only). Dark-shaded bars indicate ε values in the absence of antidote, light-shaded bars in the presence of ADH8-1 antidote and hatched bars in the presence of cADH8-1 antidote.
(A) Buffer
(B) Serum
(C) Plasma

### SEQUENCE LISTINGS

The sequence listings according to the present application include those as follows:

| | |
|---|---|
| DH6-1 | 5′ p CTG GGT TGG TGA GGT TGG TCA GCA CGG |
| | TTG GTG AGG TTG GTG TG 3′ |
| DH8-1 | 5′ p GCT GTG GTT GGT GAG GTT GGC AGC GCA |
| | CTG GTT GGT GAG GTT GGG TGC 3′ |
| DH10-1 | 5′ p GCA TGT GGT TGG TGA GGT TGG CAT GCG |
| | CTA CTG GTT GGT GAG GTT GGG TAG C 3′ |
| DH12-1 | 5′ p GCG ATG TGG TTG GTG AGG TTG GCA TCG |
| | CGA CTA CTG GTT GGT GAG GTT GGG TAG TC 3′ |
| TS1-1 | 5′ p GCT GTG GTT GGT GAG GTT GGC AGC AGC |
| | CAA GGT AAC CAG TAC AAG GTG CTA AAC GTA |
| | ATG GCT TCG GCT 3′ |
| TT4-1 | 5′ pGAG TCC GTG GTA GGG CAG GTT GGG GTG |
| | ACT CGC TGT GGT TGG TGA GGT TGG CAG C 3′ |
| TT4-2 | 5′ pGAG TCC GTG GTA GGG CAG GTT GGG GTG |
| | ACT CGC TGT GGT TGG TGA GGT TGG ACA GC 3′ |
| TT4-3 | 5′ pGAG TCC GTG GTA GGG CAG GTT GGG GTG |
| | ACT CGC TGC GGT TGG TGA GGT TGG GCA GC 3′ |
| DH8-Br1 | 5′ pGCT GTG GTT GGB GAG GBB GGC AGC GCA |
| | CBG GBB GGB GAG GBB GGG BGC 3′ |

where B=5-bromo-2′-deoxyuridine, 5-iodo-2′-deoxyuridine or other photoactive nucleotide analogue.

| | |
|---|---|
| ADH8-1 | 5′ pGCA CCC AAC CTC ACC AAC CAG TGC GCT GCC |
| | AAC CTC ACC AAG CAC AGC 3′ |

### DETAILED DESCRIPTION OF INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. The subject specification contains nucleotide sequence information prepared using the programme PatentIn Version 3.0, presented herein after the references. Each nucleotide sequence is identified in the sequence listing by the numeric indicator <210> followed by the sequence identifier (e.g. <201>1, <210>2, etc). The length, type of sequence (eg DNA) and source for each nucleotide sequence are indicated by information provided in the numeric indicator fields <211>, <212> and <213>, respectively. Nucleotide sequences referred to in the specification are defined by the information provided in numeric indicator field <400> followed by the sequence identifier (e.g. <400>1, <400>2, etc).

The reference to any prior art in this specification is not, and should not be taken as, an acknowledgment or any form of suggestion that that prior art forms part of the common general knowledge in Australia.

As referred to herein, “a target binding region” is a region within an aptamer that binds to a desired target (eg cell, protein or other molecule) and thus includes a molecular recognition region within the aptamer which can bind to a target. The terms “region” and “domain” as used herein may be used interchangeably.

In a preferred aspect the present invention relates to an aptamer comprising a circular oligonucleotide defining one to four thrombin binding quadruplex regions. The aptamers of the present invention therefore include oligonucleotides that specifically bind equivalently or non-equivalently to molecules such as thrombin and may optionally include sequence motifs that may specifically bind other elements such as cells, cellular components or other materials such as biomolecules, chromatography columns or beads or the like. The term “oligonucleotide” is intended to encompass nucleic acids including not only those with conventional bases, sugar residues and internucleotide linkages, but also those that may contain modifications of any or all of these components. As referred to herein, oligonucleotides therefore include RNA or DNA sequences of two or more nucleotides in length, (unless the context requires otherwise) and may specifically include short sequences such as dimers or trimers which may be intermediates in the production of aptamers according to the invention. Oligonucleotides as mentioned herein encompass those in single chain or duplex form and also specifically include those having quadruplex regions, for example of the type characterised by linked guanine quartets such as exemplified in FIG. 1A. The oligonucleotides forming the aptamers of the present invention may constitute DNA (polydeoxyribonucleotides containing 2′-deoxy-D-ribose or modified forms thereof), RNA (polyribonucleotides containing D-ribose or modified forms thereof) or any other type of polynucleotide which is an N-glycoside or C-glycoside of a purine or pyrimidine base, or modified purine or pyrimidine base.

The oligonucleotides according to the present invention may be formed of conventional phosphodiester-linked nucleotides and synthesised using standard solid phase (or solution phase) oligonucleotide synthesis techniques or enzymatic synthesis techniques (with or without primer), which are well known to those skilled in the art. It is also possible, however, for the oligonucleotides of the invention to include one or more “substitute” linkages as would be well understood in the art. Substitute linkages of this type may for example include phosphorothioate, phosphorodithioate or phosphoramidate type linkages or other modified linkages that would be well understood by persons skilled in the art.

The term “nucleoside” or “nucleotide” encompasses ribonucleosides or ribonucleotides, deoxyribonucleosides or deoxyribonucleotides, or other nucleosides which are N-glycosides or C-glycosides of a purine or pyrimidine base, or modified purine or pyrimidine base. Thus, the stereochemistry of the sugar carbons may be other than that of D-ribose in one or more residues. Analogues where the ribose or deoxyribose moiety is replaced by an alternative structure such as for example a 6-membered morpholino ring as described in U.S. Pat. No. 5,034,506 or where an acyclic structure serves as a scaffold that positions the base analogues are also encompassed. Elements ordinarily found in oligonucleotides such as the furanose ring or the phosphodiester linkage may be replaced with any suitable functionally equivalent element and modifications in the sugar moiety, for example wherein one or more of the hydroxyl groups are replaced with halogen, or aliphatic groups or are functionalised as ethers, amines and the like, are also included.

The nucleosides and nucleotides of the oligonucleotides according to the invention may contain not only the natively found purine and pyrmidine bases A, T, C, G and U, but also analogues thereof, which will generally be referred to as “nucleotide analogues”. Nucleotide analogues may for example include alkylated purines or pyrimidines, acylated purines or pyrimidines or other heterocycles. The nucleotide analogues encompassed by the present invention are those generally known in the art, many of which are used as chemotherapeutic agents, and examples of which include 7-deazadenine, 7-deazaguanine, pseudoisocytosine, N⁴,N⁴-ethanocytosine, 8-hydroxy-N⁶-methyladenine, 4-acetylcytosine, 5-(carboxyhydroxylmethyl)uracil, 5-fluorouracil, 5-bromouracil, 5-carboxymethylaminomethyl-2-thiouracil, 5-carboxymethylaminomethyl uracil, dihydrouracil, inosine, N⁶-isopentenyl-adenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyl uracil, 5-methoxy aminomethyl-2-thiouracil, beta-D-mannosylqueosine, 5′-methoxycarbonylmethyluracil, 5-methoxyuracil, 2-methylthio-N⁶-isopentenyladenine, uracil-5-oxyacetic acid methyl ester, pseudouracil, 2-thiocytosine, 5-methyl-2-thiouracil, 2-thiouracil, 4-thiouracil, 5-methyluracil, N-uracil-5-oxyacetic acid methylester, uracil-5-oxyacetic acid, queosine, 2-thiocytosine, 5-propyluracil, 5-propylcytosine, 5-ethyluracil, 5-ethylcytosine, 5-butyluracil, 5-butylcytosine, 5-pentyluracil, 5-pentylcytosine, and 2,6-diaminopurine. In certain circumstances there may be a call for photoactive analogues which will degrade on exposure to radiation at an appropriate energy. Examples of photoactive analogues include 5-bromo-2′-deoxyuridine and 5-iodo-2′-deoxyuridine. In other circumstances there may be call for fluorescent nucleotide analogues to enable detection by fluorescence microscopy, fluorescence resonance energy transfer (FRET) or other fluorescence detection methodologies known to those skilled in the art. In yet other circumstances, there may be call for electrochemically-labelled nucleotide derivatives to enable detection by electrochemical methods. Examples of electrochemically-labelled nucleotides include ferrocenyl- and metal complex derivatives of any nucleotide moiety including 2′-deoxyuridine. The sugar residues of the oligonucleotides of the invention may be other than conventional ribose and deoxyribose residues and may for example contain analogous forms of ribose or deoxyribose sugars as are well understood in the art. Particular possibilities include sugars substituted at the 2′-position of the furanose residue.

As explained above preferred aptamers according to the present invention define one to four thrombin binding quadruplex regions. In a preferred embodiment of the invention the aptamers define two, three or four thrombin binding quadrupled regions, in which case the quadrupled regions are separated by at least partially duplex regions. That is, within the circular oligonucleotide, regions of complementarity that demonstrate base pairing are located between each of the thrombin binding quadruplex regions. Preferably, the aptamers of the invention comprise two or three, most preferably two thrombin binding quadruplex regions. In the situation where the aptamer comprises only a single thrombin binding quadruplex region it is preferred that the aptamer includes one or more binding domains that bind cells or cell components or other materials.

By the term “thrombin binding quadruplex region” it is intended to encompass a nucleotide sequence having a core of two guanine quartets which exhibits specific binding to thrombin. Examples of the nucleotide sequences that define thrombin binding quadruplex regions include the consensus sequence d(GGTMGGXGGTTGG), where M represents A or T and X represents a sequence of any two to five nucleotides or nucleotide analogues. In preferred embodiments X may represent TGT, GCA or TGA. A more specific example is the 15-mer d(GGTTGGTGTGGTTGG), also known as GS-522, as shown in FIG. 1A. Another example of a thrombin binding quadruplex region is the sequence d(GGTAGGGCAGGTTGG) (<400>13) which binds at the heparin-binding exosite (exosite 1). Methods of identifying specific thrombin binding oligonucleotides are for example provided within WO 92/14842, the disclosure of which is included herein in its entirety by way of reference.

Within the aptamers of the invention wherein there are two, three or four thrombin binding quadruplex regions or where there is a single thrombin binding quadruplex region and one or more cellular, cell component or material binding domains, the various binding regions/domains are preferably separated by at least partially duplex regions. By this it is intended to convey that within the circular aptamer, and between the various binding regions/domains there is a sequence of at least two nucleotides complimentary to a sequence of at least two nucleotides from another section of the aptamer, which complimentary sequences are configured to allow base pairing and thereby the formation of oligonucleotide that is duplex in the complimentary sections. Preferably each chain of the duplex regions includes two to fifty, more preferably two to twenty and particularly preferably four to twelve nucleotides and/or nucleotide analogues.

In another aspect the invention relates to aptamers that may be utilised to produce circular aptamers according to the invention. In this regard the invention also includes single stranded oligonucleotides wherein 5′ and 3′ termini may be ligated to produce a circular aptamer. Of course, the non-circular aptamers of this type should include all the necessary components of the aptamers of the invention, namely one to four thrombin binding quadruplex regions and the optional cellular or other material binding domains, in addition to nucleotide sequences that will define the at least partially duplex regions between the thrombin binding quadruplex regions and cellular, cellular component or other material binding domains if present, when the termini are ligated. Preferably the non-ligated aptamers are phosphorylated at their 5′ end to thereby provide the functionality required for enzymatic and/or chemical ligation.

Ligation may involve preferably enzymatic or alternatively chemical closure of a phosphorylated open chain oligonucleotide in which the ends are held together by base pairing to a complimentary template sequence (Kool, 1996). Template directed approaches such as this are generally utilised for cyclisation of oligonucleotides greater than thirty nucleotides in length (Dolinnaya et al, 1993; Prakash and Kool, 1992). Exemplary chemical ligation techniques include the use of a condensing agent such as cyanogen bromide or carbodiimide (Dolinnaya et al, 1988; 1991; 1993; Kool, 1991; Fedorova et al, 1995). For example, enzymatic ligation may be performed using standard conditions for T4 DNA ligase (Dolinnaya et al, 1988) and circularised DNAs may be purified by use of denaturing polyacrylamide gel electrophoresis (PAGE).

It is also possible for other approaches to be adopted in synthesis of cyclic oligonucleotides including solution methods (Rao and Reese, 1989; Capobianco et al, 1990), polymer supported methods (De Napoli et al, 1993) and template-directed approaches (Kool, 1991; Rumney and Kool, 1992; Dolinnaya et al, 1993). In the past solution phase approaches have been utilised to synthesise small and medium sized oligonucleotides (for example less than 10 nucleotides in length) and solid phase processes have been utilised to produce medium sized cyclic oligonucleotides (for example ten to thirty nucleotides in length).

According to the present invention it is preferred for the oligonucleotides of the invention to be prepared utilising a self-templating approach with oligonucleotides that have internal base pairing (Erie et al, 1989; Ashley and Kushlan, 1991). This self-templating approach preferably involves the enzymatic and/or chemical ligation of the duplex region of the aptamer which is formed upon folding.

As previously discussed the aptamers according to the present invention may include one or more cellular, cell component or other material binding domains which may for example offer utility in assisting uptake across the gastrointestinal tract or targeting the aptamers to specific cell types and may offer advantages in linkage to materials such as implantable biomaterials, components of blood or blood product storage or transfer equipment and diagnostic or filtration equipment components. For example, aptamers of the present invention can be targeted to bind to any of the CD (cluster of differentiation) antigens of which there are 166 presently known, specific examples of which include L-selectin (CD62L); CD41 and CD42 (located on platelets) and CD44 on leukocytes. In one preferred embodiment of the invention the circular aptamer includes a domain with binding affinity for L-selectin, a surface protein found on cells in the circulation, particularly leukocytes (Bradley et al, 1992). An advantage that may be associated with aptamers having an L-selectin binding domain is that they can be anchored to circulating cells which may result in the aptamer being retained within the systemic circulation. A further advantage arises in capture of leukocytes from physiological fluids, especially blood. L-selectin DNA aptamers can be generated by in vitro selection methods as discussed in Hicke et al (1996), the disclosure of which is included herein in its entirety by way of reference. Three L-selectin aptamers produced according to the methods of Hicke et al (1996), namely LD201, LD174 and LD196, were modified by removal of bases from each end to generate preserved duplex regions and were attached to the 3′-end of the quadruplex-duplex thrombin aptamers to produce the TS1-1 sequence (amongst others) as referred to above. While the L-selectin aptamers LD201, LD174 and LD196 have little sequence homology they bind L-selectin with comparable nanomolar affinities.

An example of another binding motif that may be incorporated within the aptamers of the invention to provide selective binding to cells is the motif for binding to the cell-surface oligosaccharide cellobiose, as described in Yang et al, 1998, the disclosure of which is included herein in its entirety by way of reference.

In a particularly preferred embodiment oligonucleotides of the formula I are utilised to form the circular aptamers according to the invention, wherein formula I is as follows:
5′ D₁′wQxD₁D₂yQzD₂′ 3′ Formula I

Within formula I the regions defined as “Q” represent thrombin binding quadruplex regions having nucleotide sequence GGTMGGXGGTTGG, where M represents A or T and X represents a sequence of two to five nucleotides and/or nucleotide analogues. In this context it is preferred that X represents TGT, GCA or TGA.

Within formula I the variables w, x, y and z may be the same or different and can represent a sequence of zero to ten nucleotides and/or nucleotide analogues. These variables are intended to represent additional or extraneous nucleotides and/or nucleotide analogues not directly within the thrombin binding quadruplex regions and not necessarily internally complementary. The nucleotides represented by w, x, y and z therefore attribute to bulges or bunching within the circular aptamer and may play a role in directing the orientation of the thrombin binding quadruplex regions. It is preferred for w, x, y and z to represent, independently, zero to four nucleotides and/or nucleotide analogues and it is more particularly preferred for them to represent just zero or one nucleotide or nucleotide analogue. It is most preferred for one, two, three or four of w, x, y and z to represent a single nucleotide, which is most preferably T.

The D₁ and D₂ variables may be the same or different and each represent a sequence of zero to twenty-five nucleotides and/or nucleotide analogues, with the proviso that D₁ and D₂ together comprise at least two nucleotides or nucleotide analogues. It is preferred for D₁ and D₂ together to represent two to twenty nucleotides and/or nucleotide analogues, more preferably four to twelve nucleotides and/or nucleotide analogues.
The variables D₁′ and D₂′ may be the same or different and each represent a sequence of zero to fifty nucleotides and/or nucleotide analogues. However, at least two consecutive nucleotides or nucleotide analogues of D₁′ and/or D₂′ are complementary to at least two consecutive nucleotides and nucleotide analogues of D₁ and/or D₂, so as to allow duplex formation between complementary nucleotides or nucleotide analogues. Although it is preferred for the aptamers of the invention to be somewhat symmetrical in the sense that D₁, D₂, D₁′ and D₂′ are of the same or at least similar nucleotide length, this is by no means essential. For example, it is possible for D₁′ to be two nucleotides in length while D₂′ is four nucleotides in length and that these six nucleotides are complementary to six nucleotides defined by D₁ and D₂ in combination.

As it is intended for D₁′ and D₂′ or at least elements of them to be complementary with D₁/D₂ or at least elements of the combination, the sense of these elements needs to be reversed to allow complementarity by folding. Specific examples of respective pairs of D₁ and D₁′ include CAG and CTG; CAGC and GCTG; CATGC and GCATG; CATCGC and GCGATG and specific examples of D₂ and D₂′ include CAC and GTG; GCAC and GTGC; GCTAC and GTAGC; GACTAC and GTAGTC. A diagrammatic representation of an aptamer of the present invention, having two thrombin binding quadruplex regions (T) is shown in FIG. 1B.

Another aspect of the invention relates to antidote (or antisense) oligomers of aptamers of the invention. These may also be referred to herein as “antiaptamers”. Antidote oligomers (or antiaptamers) can counteract the effect of the corresponding aptamer and thus may be useful in circumstances where the effect of the aptamer is greater than desired, for example by using too much aptamer. The antiaptamers are preferably at least 10 nucleotides and/or nucleotide analogues in length and are complementary to a sequence of at least 10 nucleotides and/or nucleotide analogues of an aptamer of the invention.

One embodiment of this aspect relates to antidotes of the thrombin binding aptamers which comprise aptamers of at least ten nucleotides and/or nucleotide analogues in length which are complementary to a sequence of at least ten nucleotides and/or nucleotide analogues from within a thrombin binding aptamer of the invention. Preferably, the region of complementarity of the antisense sequence encompasses at least a portion of one or more of the thrombin binding quadruplex regions. More preferably, the antidote aptamers are complementary to at least a portion of each of the thrombin binding quadruplex regions and particularly preferably the antidote aptamers constitute an antisense oligonucleotide to the entire sequence of the thrombin binding aptamer of the invention. A diagrammatic representation of an antidote aptamer is shown in FIG. 1C.

The invention thus also provides a method for counteracting the effect of an aptamer of the invention comprising contacting the aptamer with a counteracting effective amount of its antiaptamer.

As used herein, “counteracting” refers to the inhibition, halting or partial or full reversal of the effect of the aptamer.

Aptamers according to the present invention and their antisense antidotes may be formulated into standard pharmaceutical dosage forms by combination with one or more pharmaceutically acceptable carriers and/or excipients. Examples of pharmaceutically acceptable carriers and excipients are provided within Remington's Pharmaceutical Sciences, 17th Edition, Mack Publishing Co, Easton, Pa., USA, the disclosure of which is included herein in its entirety, by way of reference. Although it is preferred for the aptamers of the present invention to be formulated into oral dosage forms, it is additionally possible for formulation into forms suitable for intravenous, intramuscular, subcutaneous, buccal, intraperitoneal, rectal, vaginal, nasal and ocular delivery, for example.

As used herein “pharmaceutically acceptable carrier and/or excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients may also be incorporated within the compositions of the invention. Dosage forms according to the present invention which may for example be formulated as tablets, troches, pills, capsules, injectables, salves, ointments, drops, sprays, powders and the like will preferably be formulated into unit dosage forms, which may for example contain between about 0.1 μg and 2,000 mg of active compound. As will be well recognised by a skilled medical practitioner or pharmacist the- effective dosage of the active ingredients according to the present invention will be dependent upon the nature of the disorder being treated and the height, age, weight, sex and general fitness of the patient concerned.

The aptamers according to the present invention are particularly suited for treatment and/or prevention of thrombosis, stroke, myocardial infarction and respiratory failure. The aptamers according to the invention may also be utilised in prevention of clotting as a result of trauma, and may be used in surgery, in the treatment and/or prevention of inflammatory disorders, cancer metastasis, neural disease and blood coagulation. In the case where rapid reversal of action of the aptamers according to the invention is required, for example in the situation of an overdose, it is possible to administer the antidote aptamers in an amount sufficient to bind the thrombin binding aptamers and competitively inhibit their activity.

The aptamers according to the present invention may also be utilised in the prevention of blood or blood product coagulation by their incorporation within or addition to blood sample tubes and bags or other materials that blood or blood products such as serum or plasma may come into contact with. In preferred embodiments of the invention aptamers including material binding domains may be incorporated into materials such as implantable biomaterials including stents, prostheses and the like to prevent localised blood clotting. The aptamers may also be utilised in conjunction with tissue and/or organ transplants and/or xenotransplants, particularly in relation to vascular grafts. The aptamers according to the invention may also be utilized in the capture of leukocytes from physiological fluids, especially blood, as part of a medical or genetic diagnostic procedure.

The invention will now be further described with reference to the following non-limiting examples.

### EXAMPLES

### Example 1

### Preparation, Circularisation and Isolation of Aptamers

Materials

General Reagents

N-2-hydroxy-ethylpiperazine-N′-2-ethane (HEPES, Sigma Chemical Co.), spermidine (Sigma Chemical Co), tris acetate (BDH), 2(N-morpholino)ethanesulfonic acid (MES; Sigma Chemical Co.), 3,3′-deithyl-9-methyl-4,5,4′5′-dibenzothiacarbocyanine (STAINS-ALL; Sigma Chemical Co.), TE-saturated phenol/chloroform pH 8 (Progen), Dithiothreitol (DTT; Progen), ammonium persulphate (APS; Sigma Chemical Co.), boric acid (BDH), potassium chloride (BDH), Tris (Ajax Chemicals), magnesium chloride (BDH), calcium chloride (BDH), glycerol (Ajax Chemicals), β-mercaptoethanol (Ajax Chemicals) and adenosine 5′ triphosphate (ATP; Sigma Chemical Co.). Bio-Spin P6 and P30 columns, N,N,N′N′-tetramethylethylenediamine (TEMED), 40% bisacrylamide solution and ethidium bromide were purchased from Bio-Rad Laboratories. SYBR Green II RNA stain was purchased from Molecular Probes. All reagents were of analytical grade and all solutions were prepared with Milli-Q deionised water.

Enzymes and Associated Materials

Calf intestine alkaline phosphatase (MBI Fermentas), human α-thrombin 3700 U/mg (Sigma Chemical Co.), bovine α-thrombin 5000 U (Armour Pharmaceutical Co.), T4 DNA Ligase (MBI Fermentas) and cyanogen bromide (Sigma Chemical Co.) were used as received.

DNA Oligonucleotide Sequences

Sequences for the quadruplex-duplex thrombin aptamers were developed from oligonucleotides studied by Macaya et al. (1995). MFOLD (Zucker, 1994) was used to determine sequence secondary structure. Set A contains the classic aptamer GS-522 and thrombin circle (TC) family; Set B contains the double header (DH) family; and Set C contains the thrombin-selectin (TS) familyoligonucleotides. A modified version of DH8 was synthesised with 5-bromo-2′-deoxyuridine phosphoramidite (Glen Research) substituted for six T residues (B=5-bromo dU). DH8-Br was used for photocrosslinking. All oligonucleotides except GS-522 were phosphorylated at the 5′-end using phospholink reagent (Perkin Elmer). Oligonucleotides were deprotected and gel purified before use.

| | | |
|---|---|---|
| SET A | | |
| GS-522 | | 15-mer |
| | 5′ | GGT TGG TGT GGT TGG 3′ |
| TC1 | 5′-P | 27mer |
| | 5′ | p ACT GGT TGG TGA GGT TGG GTG CGA AGC |
| | | 3′ (<400>14) |
| TC1-T | 5′-P | 26mer |
| | 5′ | p ACG GTT GGT GAG GTT GGG TGC GAA GC |
| | | 3′ (<400>15) |
| TC3 | 5′-P | 28mer |
| | 5′ | p ACT GGT TGG TGA GGT TGG GTG CGA AAG |
| | | C 3′ (<400>16) |
| SET B | | |
| DH6-1 | 5′-P | 44mer |
| | 5′ | p CTG GGT TGG TGA GGT TGG TCA GCA CGG |
| | | TTG GTG AGG TTG GTG TG 3′ |
| DH8-1 | 5′-P | 48mer |
| | 5′ | p GCT GTG GTT GGT GAG GTT GGC AGC GCA |
| | | CTG GTT GGT GAG GTT GGG TGC 3′ |
| DH10-1 | 5′-P | 52mer |
| | 5′ | p GCA TGT GGT TGG TGA GGT TGG CAT GCG |
| | | CTA CTG GTT GGT GAG GTT GGG TAG C 3′ |
| DH12-1 | 5′-P | 56mer |
| | 5′ | p GCG ATG TGG TTG GTG AGG TTG GCA TCG |
| | | CGA CTA CTG GTT GGT GAG GTT GGG TAG |
| | | TC 3′ |
| SET C | | |
| TS1-1 | 5′-P | 69-mer |
| | 5′ | p GCT GTG GTT GGT GAG GTT GGC AGC AGC |
| | | CAA GGT AAC CAG TAC AAG GTG CTA AAC |
| | | GTA ATG GCT TCG GCT 3′ |
| TS2-1 | 5′-P | 69mer |
| | 5′ | p GCT GTG GTT GGT GAG GTT GGC AGC AGC |
| | | TGG CGG TAC GGG CCG TGC ACC CAC TTA |
| | | CCT GGG AAG TGA GCT 3′ (<400>17) |
| TS3-1 | 5′-P | 69mer |
| | 5′ | p GCT GTG GTT GGT GAG GTT GGC AGC AGC |
| | | CAT TCA CCA TGG CCC CTT CCT ACG TAT |
| | | GTT CTG CGG GTG GCT 3′ (<400>18) |

MODIFIED OLIGONUCLEOTIDE

| | | |
|---|---|---|
| DH8-Br1 | 5′-P | 48mer |

B = 5-bromo-2′-deoxyuridine

| | |
|---|---|
| 5′ | p GCT GTG GTT GGB GAG GBB GGC AGC GCA |
| | CBG GBB -GGB GAG GBB GGG BGC 3′ |

Methods
Sterilisation of Materials

All heat labile solutions were sterilised by filtration through 0.2 μm cellulose acetate disposable filters (Millipore). All other solutions were sterilised by autoclaving for 30 min (1.0 kg cm⁻², 120° C.). Disposable microfuge tubes and spin columns were all sterilised by autoclaving. Biological waste was autoclaved prior to disposal. All other waste was disposed of in accordance with the regulations recommended by the UNSW Safety Unit.

Cleavage and Deprotection of Synthesised Oligonucleotides

Concentrated ammonium hydroxide solution (5 M) was applied to the synthesis column using a 1 mL syringe. Columns were inverted and several aliquots of ammonium hydroxide solution were passed through the column over a 1 h period at room temperature. The solution was then expressed into a screw cap tube and placed in a water bath at 55° C. overnight. After incubation, the tubes were dried under vacuum in a Speed-Vac SC110 (Savant Instruments Inc.) and redissolved in 100 μL sterile water. Samples were then purified by gel electrophoresis.

Phenol Extraction and Ethanol Precipitation

Protein was removed from aqueous samples by extraction with an equal volume of buffered phenol (Tris, pH 8.0). The DNA was concentrated by precipitation with ice-cold ethanol added at 2.5×the volume of aqueous sample after addition of one-tenth sample volume of 3 M sodium acetate. After mixing, samples were left at −20° C. for one hour and immediately centrifuged at 10 000×g (4° C.) for 15-20 min. Precipitated DNA was washed with 1 mL 95% ethanol and centrifuged again at 10 000×g for a further 2 min. The supernatant was removed and the pellets dried by vacuum centrifugation in a Speed-Vac SC110 vacuum concentrator.

Serum Isolation

Whole blood (20 mL) was clotted at 37° C. for 5 min. Clotting was initiated by contact with a glass slide. Serum was collected by centrifugation at 3000 rpm for 20 minutes in a Clements GS100 swing-out centrifuge. Serum samples (2 mL) were stored at −70° C. All blood products were handled in accordance with UNSW biological hazard guidelines.

Gel Electrophoresis

Gel Purification

Newly synthesised ssDNA and circular ssDNA were purified by 20% denaturing PAGE. Approximately 50-100 μg of nucleic acid was loaded onto each lane of a 10 cm×8 cm×0.15 cm gel in loading buffer not containing tracking dyes. A target product marker was also loaded with buffer containing tracking dyes in order to facilitate both estimation of running time and identification of correct products. Gels were run at a constant voltage of 100 V in 1×TBE buffer (pH 8.0) on a Mini-Protean II gel electrophoresis apparatus (Bio-Rad Laboratories). Gels were stained for 15 min in RO water (100 mL) containing ethidium bromide (0.5 μg/mL). Nucleic acids were visualised by UV shadowing and the bands excised using sterile implements. Nucleic acids were eluted from crushed gel fragments overnight by diffusion at 37° C. in a solution of 0.3 M NaCl, 10 mM Tris-HCl and 1% (v/v) phenol. Targets were collected via ethanol precipitation and dried in a vacuum concentrator. The dry samples were redissolved in sterile water and further purified in a Bio-Spin P6 column. Final nucleic acid concentrations were determined by UV spectrophotometry.

SDS PAGE

SDS PAGE consisting of a 10% resolving and 4% stacking gel was used to determine the purity and approximate quantity of protein. A stock solution containing broad range size markers was diluted 20× in SDS reducing sample buffer (0.5 M Tris-HCl, pH 6.8, 10% glycerol, 10% SDS, 0.1% bromophenol blue, β-mercaptoethanol). 10 μL protein samples were mixed in 5 μL sample buffer. All samples were heated to 90-95° C. for 5 min before loading onto the gel. Gels were run in SDS/Tris-HCl at 50 mA for 1 h. Electrophoresed gels were stained with 0.1% coomassie blue for half an hour and destained for 1-3 hours in 40% methanol/10% acetic acid and dried overnight.

Agarose Gel

Agarose gels were used to determine the activity of preparative T4 DNA ligase after purification. Lambda phage DNA standards (2 μg) and T4 DNA ligase treated samples in loading dye were run on 1% agarose gels (0.5 g agarose, 49.5 mL H₂O) in 1× TBE buffer an 100 V for 1 h. Gels were stained with ethidium bromide (0.5 μg/mL) for 30 min and visualised by UV illumination.

UV Spectrophotometry

DNA Quantitation

DNA was quantified by measuring the absorbance of a suitably diluted sample at 260 nm using a JASCO V-530 UV/VIS Spectrophotometer. Purity was gauged by the ratio of absorbance between 260 nm and 280 nm. The following calculation was used to estimate DNA concentration
[DNA]=A₂₆₀×dilution factor×33 μg/mL×sample volume
Melting Profiles

Absorbance versus temperature profiles were measured at a wavelength corresponding to the average maximum absorbance achieved at 5 and 95° C. using the JASCO v530 spectrophotometer interfaced to a PC. Melting profiles were obtained by increasing the temperature from 5-95° C. at a constant rate of 0.8° C./min with a programmable, thermoelectrically-controlled cell holder. Melting profiles of samples (OD˜0.5) were performed in 100 mM K₃PO₄ buffer pH 7.5 and circularisation buffer (1 M MES/0.02 M MgCl₂, pH 7.5). First derivatives of melting curves were used to calculate melting temperature (Tₘ).

Mass Spectroscopy

Oligonucleotide 5′-phosphorylation was determined using MALDI-TOF mass spectrometry (The Voyager). Oligonucleotides were first desalted using AGSOW-X8 NH₄⁺ resin (Bio-Rad). Sterile water (0.5-1 μL) containing of each oligonucleotide (1-10 pmol) and picolinic acid matrix (0.5-1 μL of 400 mM) were mixed and then applied to a metallic target. Negative ion mass spectra were used to detect aptamers. Analysis was performed using GRAM and MONITOR software.

Circularisation

Enzymatic Ligation: T4 DNA Ligase

Oligonucleotides were heated in selection buffer (100 mM KCl, 1 mM MgCl₂, 20 mM Tris acetate, pH 7.4; Macaya et al, (1995)) to 75-85° C. and slowly cooled to 0-5° C. over 30-60 min. T4 DNA Ligase buffer (1×) and T4 DNA ligase (5 U/μg DNA) were added in the presence or absence of bovine α-thrombin (20%). The reaction mix was placed at 15-25° C. for >16 h. This was immediately followed by phenol extraction and ethanol precipitation. Ligation products were analysed on 20% PAGE.

Larger scale circularisation experiments were conducted in a similar fashion to the above procedure, however, ligations did not contain thrombin. Reactions were incubated at room temperature for periods up to 4 days. Circular products were subsequently obtained using gel purification.

Chemical Ligation: Cyanogen Bromide

This procedure was modified from the cyanogen bromide ligation described by Dolinnaya et al. (1993) and Fedorova et al. (1995). Oligonucleotide in 10× vol buffer (0.25-0.5 M MES-(C₂H₅)₃N, pH 7.5, 0.02 M MgCl₂, with or without 50 mM KCl) was heated to 85° C. for 2 min and slowly cooled to 0-5° C. over 30 min. BrCN in acetonitrile (5 M) was added to the samples on ice at one-tenth of the volume of the reaction mix. Final concentrations of the oligonucleotide and BrCN were 50 μM and 0.5 M, respectively. Reactions were incubated on ice for 5 min. Upon completion, the reaction was quenched by addition of 2.5× vol of 100% ice-cold ethanol. Samples were ethanol precipitated and analysed by 20% denaturing (urea) PAGE. This method was used in both small and large scale circularisation procedures.

Exonuclease Treatment of Circular Product

Pellets from circularisation experiments were redissolved in T4 DNA polymerase buffer. T4 DNA polymerase (6 Units/μg DNA) was added to the samples and reaction tubes were placed at 25° C. for 24 h. Protein was removed by phenol extraction followed by ethanol precipitation. Results were analysed on 20% denaturing (urea) PAGE.

### Example 2

### Thrombin Inhibition Assays

All clotting times were estimated using a fibrometer (Behring Diagnostics).

Methods

Selection Buffer

The assay for inhibition of thrombin-catalysed fibrin clot formation in serum free medium was modified from Macaya et al, (1995). Human fibrinogen in selection buffer (140 mM NaCl, 5 mM KCl, 1 mM MgCl₂, 1 mM CaCl₂, 20 mM Tris acetate, pH 7.4, 200 μL) was equilibrated at 37° for 1 min in the presence of each oligonucleotide. Reactions were initiated by the addition of bovine α-thrombin (100 μL in selection buffer preequilibrated to 37° C. for 5 min). Final concentrations of 2 mg/mL fibrinogen and 100 nM oligonucleotide were reached. Thrombin concentration varied from 50-100 nM to achieve a baseline (no oligonucleotide present) clotting time of approximately 30-40 s.

Serum

Conditions for serum assays were taken from Macaya et al. (1995). Oligonucleotides were incubated in serum (100 μL) at 37° C. for 1 min. Clotting was initiated by the addition of fibrinogen (200 μL) and thrombin (100 μL) in selection buffer pre-equilibrated to 37° C. Final concentrations of 50 nM DNA, 1.5 mg/mL fibrinogen and 50-100 nM thrombin were achieved with a baseline clotting time of between 30-40 s.

Results

Aptamer families were tested for their ability to inhibit thrombin using standard activity assays. Thrombin aptamers inhibit thrombin-activated clot formation by binding to the fibrinogen recognition site of the enzyme, preventing fibrinogen being cleaved into fibrin. Initially, anti-thrombin activity of aptamers was examined in the absence of blood products. The simplest system involves the isotonic cell- and protein-free environment of the selection buffer used for the aptamer isolation. Aptamers (100 nM) were incubated in selection buffer containing a fixed concentration of fibrinogen (2 mg/mL) at 37° C. Clotting is initiated by the addition of thrombin and the time taken for clot formation is conventionally measured using a fibrometer or coagulator. Thus, activity of the aptamer is defined in terms of clotting time.

Activity in Selection Buffer

Clotting times for each oligonucleotide family in selection buffer are presented in Table 1 and FIG. 2. These times were compared to the classic thrombin aptamer (GS-522) as positive control, and negative controls consisted of a telomere construct (H42) and an 18-mer DNA primer (P3A1). Clotting time in the presence of thrombin and fibrinogen only (no aptamer) is used as a baseline measurement.

Comparison of Aptamers

The majority of aptamers exhibited inhibition of thrombin catalysed-fibrin clot formation. With the exception of DH6-1, DH aptamers (unligated and circular) showed the greatest thrombin inhibition of the three families with clotting times at least three-fold higher than the classic aptamer GS-522 and up to ten times the baseline. Circular aptamers were generally observed to be somewhat better inhibitors than unligated species (Table 1; FIG. 2).

TABLE 1
Thrombin Inhibition by Aptamers

| | Clotting Time [s]^{a} | | | |
|---|---|---|---|---|
| | Selection buffer^{b} | | Serum^{c} | |
| Oligo | Unligated | Circular | Unligated | Circular |
| GS-522 | 161(3) | nd | 70(1) | nd |
| DH6-1 | 54(6) | 49(5) | nd | nd |
| DH8-1 | 279(16) | 449(1) | 95(7) | 352(35) |
| DH10-1 | 356(13) | 413(5) | 82(3) | 186(4) |
| DH12-1 | 374(6) | 454(44) | 121(4) | 231(3) |
| TS1-1 | 53(3) | 236(1) | 44(1) | 101(2) |

| | | |
|---|---|---|
| No Aptamer^{d} | 41(2) | 38(1) |

^{a}oligonucleotides incubated 1 min in media (selection buffer or serum) at 37° C. Tabulated values represent the averages of at least three measurements; standard errors in parentheses.
^{b}selection buffer: 100 nM DNA; 2 mg/ml fibrinogen; 2 × thrombin
^{c}serum: 50 nM DNA; 1.5 mg/ml fibrinogen; 1 × thrombin
^{d}baseline activity
nd: not determined

TC Family

Linear TC aptamers exhibited low activities (70-140 s) when compared to GS-522 (160 s). However, these clotting times are at least double the baseline time (≈40 s), indicating aptamers have thrombin inhibitory activity even though melting profiles suggest that they do not fold. The observed clotting times are believed not to be due to non-specific inhibition as TC aptamer activities are significantly more active than the negative controls (H42, P3A1). The loss of the T residue (TC1-T) had no marked effect on inhibition. The longer loop of TC-3 improved clotting time, doubling the activities of triloop oligonucleotides (TC1-T), but was not greater than the activity of GS-522.

DH Family

In most cases, unligated DH aptamers exhibited relatively high activities, 2-3 fold higher than GS-522 and at least five times higher than the baseline clotting time (FIG. 2). DH6-1 was the only unligated species not to exhibit clotting inhibition. Thrombin inhibition also generally increased with increasing duplex length and melting temperature such that the following trend was observed: DH6-1<<DH8-1<DH10-1<DH12-1. However, activity and thermal stability (Tm) tended to plateau at longer duplex sizes (DH10-1 and DH12-1; FIG. 2).

Circularised species (except cDH6-1) increased baseline clotting time more than 10-fold (450 s cf. 40 s) and displayed at least three times the activity of the classic aptamer GS-522 (FIG. 2). Given standard error in the activities of cDH8-1, cDH10-1 and cDH12-1 (Table 1) differences in clotting times are not considered to be significant. Unlike other cDH aptamers, cDH6-1 did not inhibit clotting and exhibited similar activity to the negative controls.

TS Family

Of the linear TS species, only TS2-1 demonstrated significant thrombin inhibition (t≈70 s), however this is less than half the activity of GS-522. Both TS1-1 and TS3-1 exhibited clotting inhibitions similar to the negative controls. Circularisation increased activity of linear TS1-1 by 200 s. The clotting inhibition of cTS1-1 is two-fold higher than GS-522 and similar to unligated DH8-1.

Activities in Serum Supplemented Media

To investigate the potential use of aptamers as anticoagulants in mammals, the ability of these oligonucleotides to inhibit thrombin in vitro using serum supplemented media was examined. Serum (pre-clotted cell-free fluid) simulates to a certain extent in vivo conditions by providing molecules and proteins, such as exo- and endo-nucleases, required for a more complete (although more complex) analysis of aptamer activity. Human serum is used in this study rather than 10% fetal calf serum (FCS; Macaya et al., 1995) to provide a better assessment of aptamer performance in the intended target species.

Clotting assays using serum (Macaya et al., 1995) were performed in a similar way to the previous assay, however, aptamers were incubated in serum for 1 min at 37° C., before fibrinogen and thrombin addition. Final concentrations of DNA and fibrinogen were also reduced (50 nM DNA; 1.5 mg/ml fibrinogen cf. selection buffer) due to limited reagents. However, the effect of serum on aptamers can be generally examined and compared to selection buffer.

As can be seen in Table 1, circular aptamers are better inhibitors of thrombin in serum, with at least two-fold higher activities than their unligated counterparts. This higher activity is not as significant in selection buffer (except cTS1-1 as linear TS1-1 exhibited no activity in either medium). cDH8-1 has a much higher anti-thrombin activity than the other cDH aptamers (350 s cf.<240 s), which is not observed for unligated DH8-1 in serum. All unligated DH oligonucleotides have similar serum clotting times. As the standard error for cDH8-1 is large, it is suspected that the actual activity may be lower than the tabled value.

### Example 3

### Serum Stability

Methods

Functional Stability Assay

Oligonucleotides were incubated in human serum (500 μL) at 37° C. and 100 μL samples were taken at 1 min and at 1, 6, 12 and 24 h. Samples were assays by the addition of fibrinogen (200 μL in selection buffer; 37° C.) followed by bovine thrombin (100 μL in selection buffer; 37° C.) to initiate the clotting reaction. Final concentrations of reagents were: 50 nM oligonucleotide, 1.5 mg/mL fibrinogen and 50-100 nM thrombin to achieve a baseline clotting time of between 30-40 s.

Physical Stability: PAGE

Oligonucleotides (2 μg) were added to serum (100 μL) and incubated at 37° C. At different time intervals 20 μL samples were taken and the reaction quenched with 20 μL phenol/chloroform pH 8. An aliquot (2× vol) of Tris-HCl (10 mM, pH 8) was also added before vortexing thoroughly. Samples were centrifuged at 14 000 rpm, 4° C. for 5 min and the aqueous layer removed. The phenol layer was re-extracted with Tris-HCl. Combined aqueous layers were ethanol precipitated and run of 20% native or denaturing PAGE. Gels were stained with SYBR Green II (1:10 000 1× TBE) for 30 min. Gels were then subjected to image analysis.

Image Analysis

PAGE gels from serum stability studies were analysed using Fluoro-S Multi-Imager (Bio-Rad). UV scanning illumination (320 nm) was used with the lens aperture fully open. Analysis of gel images was performed using Multi-Analyst/Macintosh software Version 1.0 (Bio-Rad).

Results

To investigate the susceptibility of circularised aptamers to nucleolytic activity, oligonucleotides were incubated in serum and examined for both functional and physical stability. Functional stability describes the ability of oligonucleotides to maintain their inhibition of thrombin-catalysed fibrin clot formation over time. Physical stability refers to actual nuclease degradation of aptamers as demonstrated by PAGE. Note that serum stability measurements were only taken for those unligated oligonucleotides that were circularised in high yields and exhibited significant thrombin inhibition in selection buffer (ie. DH8-1, DH10-1, DH12-1 and TS1-1).

Functional Stability

Aptamers were incubated in serum at 37° C. and their activity analysed at 1 min and 1, 6, 12 and 24 h. The results for unligated and circular aptamers are shown in FIG. 3. The classic aptamer GS-522 displayed no activity after one hour in serum, which is consistent with previous reports on its low serum stability (Griffin et al., 1993; Macaya et al., 1995). In FIG. 3A, unligated TS1-1 exhibits very little thrombin inhibition, similar to its performance in selection buffer. DH unligated aptamer activities decline over the first 6 hours to an activity approximately equal to the initial clotting time of GS-522. Differences in clotting inhibition of unligated DH oligonucleotides over the 24 h period are not thought to be significant. At 12 to 24 h there is little aptamer activity observed, however, activities do not reach baseline levels within the 24 h period.

Circular aptamers (except cTS1-1) maintained significant clotting inhibition over 24 h (FIG. 3B). At 24 h, cDH oligonucleotides exhibited clotting times equal to (cDH10-1, cDH12-1) and greater than (cDH8-1) the 1 min clotting time of the classic aptamer GS-522. cDH8-1 demonstrated the greatest clotting inhibition at each time point, with values approximately twice those of cDH10-1 and cDH12-1. The latter aptamer pair had similar activity values (Table 1) which were about four fold higher than cTS1-1. However, as this activity of cDH8-1 is significantly greater than that observed in selection buffer (taking concentrations into account), experimental error is suspected (Table 1).

The activity of all the circular aptamers showed a similar pattern of functional decay, which was different to the functional decay observed for the unligated species. In FIG. 3B, a rapid decrease in activity in the first hour is followed by a slow decline to 24 h. Beyond the first hour, aptamer activity decay corresponds well to first order kinetics and half-lives were determined using first order rate constants (Table 2). Unligated and circular DH aptamers tend to have similar functional half-lives.

According to the kinetic data, unligated and circular DH aptamers maintain their activity in serum up to seventy times longer than GS-522. The half-life of cTS1-1 is somewhat dubious since determination of half-lives requires 2-4 half-lives to be followed to ensure accuracy. The half-life of unligated TS1-1 could not be determined as it showed poor initial activity.

TABLE 2
Kinetics of Functional Decay

| APTAMER | k₁ (s⁻¹)^{a} | k₂ (s⁻¹)^{b} | t_{1/2} (h)^{c} |
|---|---|---|---|
| pDH8-1 | 2.7 × 10⁻⁵ | 9.9 × 10⁻⁶ | 19.4 |
| pDH10-1 | 2.8 × 10⁵ | 1.3 × 10⁻⁵ | 15.2 |
| pDH12-1 | 3.2 × 10⁻⁵ | 1.1 × 10⁻⁶ | 18.3 |
| pTS1-1 | 3.8 × 10⁻⁵ | —d | —d |
| cDH8-1 | 9.0 × 10⁵ | 9.1 × 10⁻⁶ | 21.1 |
| cDH10-1 | 8.6 × 10⁻⁵ | 1.4 × 10⁻⁵ | 13.7 |
| cDH12-1 | 9.6 × 10⁻⁵ | 1.2 × 10⁻⁵ | 15.7 |
| cTS1-1 | 1.3 × 10⁻⁴ | 4.5 × 10⁻⁶ | 42.7 |

^{a}Initial rate constant k₁ is determined from the slope of a linear plot of ([A]ₜ − [A]₀ vs. time (two data points).
^{b}The rate constant k₂ is determined from the slope of a plot of In([A]ₜ/[A]₀) vs (4 data points).
^{c}Half-life, t_{1/2} = In2/k₂
dnot determined

Physical (Nuclease) Stability

Physical analysis was performed using PAGE to investigate whether the initial high activity loss in the first hour of the functional assay was due to physical degradation or to another factor such as non-specific protein binding. Circular aptamers were incubated in serum at 37° C. and sampled at different time points (1 min, 1, 2, 6, 12 and 24 h). PAGE results for cDH aptamers and cTS1-1 (FIG. 4) suggest physical degradation is not responsible for the initial sharp fall in activity (FIG. 3B). Little degradation (unligated product) can be observed in the first hour and there is significant circular product at 6 (cTS1-1) and 24 h (cDH aptamers), indicating that circular aptamers are extremely stable in comparison to GS-522. The length of the duplex region was found to influence nuclease stability as cDH10-1 and cDH12-1 were degraded more quickly than cDH8-1 (FIG. 4A, C). Unligated DH12-1 was also tested for physical stability and found to be more susceptible to nuclease activity, degrading faster than cDH12-1 (FIG. 4D).

### Example 4

### ADH8-1 Antidote Aptamer Experiment

To investigate the potential of antisense hybridisation as a general aptamer antidote mechanism, the pADH8-1 reverse complement of DH8-1 was prepared. This construct contained an internal 8 bp duplex with two relatively unstructured C-rich heads (as depicted diagrammatically in FIG. 1C), as indicated by an experimental melting temperature of 31° C.

The unligated and circular forms of ADH8-1 displayed almost identical physical half-lives of 4 h in serum and 5 h in plasma. These values were consistent with a significant protective effect from the internal duplex, but a greater nuclease susceptibility than the DH constructs due to the absence of tightly folded head motifs.

The effect of aptamer topology on antidote effectiveness was further investigated by incubating aptamer/antiaptamer mixtures in serum for 10 min before fibrometer assay. This is a reasonable upper limit for a useful antidote effect. As shown in FIG. 5, interactions in which at least one of the partners was unligated displayed a complete antidote effect at an antidote:aptamer ratio of 2:1.

It is to be recognised that the present invention has been described by way of example only and that modifications and/or alterations thereto which would be apparent to persons skilled in the art, based upon the disclosure herein, are also considered to fall within the spirit and scope of the invention.

### REFERENCES

1. Wu, K. (1984) Pathophysiology and management of thromboembolic disorders. (Massachusetts: PSG Publishing Comp.)
2. Carney, D H. et al, (1985). Phosphoinositides in mitogenesis: neomycin inhibits thrombin stimulated phosphoinistides turnover and initiation of cell proliferation. Cell. 42:479-488.
3. Bar Shavit, R., and Wilner, K. (1986) Mediation of cellular events by thrombin. Int. Rev. Exp. Pathol. 29, 213-241.
4. Nierodzik, M, et al (1992) Effect of thrombin treatment of tumor cells on adhesion of tumor cells to platelets in vitro and tumor metastasis in vivo. Cancer Res. 52, 3267-72.
5. Tapparelli, C, et al (1993) Synthetic low molecular weight thrombin inhibitors: molecular design and pharmacological profile. TIPS 14, 366-376.
6. Stubbs, M., and Bode, W. (1995) The clot thickens: clues provided by thrombin structure. Trends Biochem. Sci. 20, 23-28.
7. Jaques, L. (1980) Heparins-anionic polyelectrolyte drugs. Pharmacol. Res. 31, 99.
8. Topoi, E., el al, (1989) A randomized controlled trial of intravenous plasminogen activator and early intravenous heparin in acute myocardial infarction. Circulation 79,281.
9. Guschlbauer, W., et al (1990) Four stranded nucleic acid structures 25 years later: from guanosine gels to telomere DNA. J. Biomol. Strct. Dynam. 8, 491-511.
10. Padmanabhan, K., et al (1993) The structure of alpha-thrombin inhibited by a 15-mer single-stranded DNA aptamer. J. Biol. Chem. 268, 17651-654.
11. Li, W.-X., et al (1994) A novel nucleotide-based thrombin inhibitor inhibits clot-bound thrombin and reduces arterial platelet thrombus formation. Blood 83, 677-682.
12. Griffin, L., et al (1993) The discovery and characterisation of a novel nucleotide-based thrombin inhibito. Gene 137,25-31.
13. Macaya, R., et al (1995) Structural and functional characterisation of potent antithrombotic oligonucleotides possessing both quadruplex and duplex motifs. Biochem 34,4478-92.
14. Kool, E. (1996) Circular oligonucleotides: New concepts in oligonucleotide design. Ann. Rev. Biophys. Biomol. Struct. 25, 1-28.
15. Dolinnaya, N., et al (1993) Oligonucleotide circularisation by template directed chemical ligation. Nucl. Acids Res. 21, 5403-7.
16. Prakash, G., and Kool, E. (1992) Structural effects in the recognition of DNA by circular oligonucleotides. J. Am. Chem. Soc. 114, 3523-27.
17. Dolinnaya et al, 1988 Site-directed modification of DNA duplexes by chemical ligation. Nucl. Acids Res. 16, 3721-38.
18. Dolinnaya, N., et al (1991) The use of BrCN for assembling modified DNA duplexes and DNA-RNA hybrids; comparison with water soluble carbodiimide. Nucl. Acids Res. 19, 3067-72.
19. Kool, E. (1991) Molecular recognition by circular oligonucleotides. Increasing the selectivity of DNA binding. J. Am. Chem. Soc. 113, 6265-66.
20. Fedorova, O., et al (1995) Study of the mechanism of chemical ligation of DNA by cyanogen bromide. Bioorgan. Khimiia 21, 868-73.
21. Rao, M., and Reese, C. (1989) Synthesis of cyclic oligodeoxynucleotides via the filtration approach. Nucl. Acid Res. 12, 8221-39.
22. Capobianco, M., et al (1990) One pot solution synthesis of cyclic oligodeoxynucleotidees. Nucl. Acids Res. 18, 2661-69.
23. De Napoli, L., et al (1993) PEG-supported synthesis of cyclic oligodeoxynucleotides. Nucleosides.Nucleotides 12, 21-30.
24. Rumney, N., and Kool E. (1992) DNA recognition by hybrid oligoether-oligodeoxynucleotide marcrocyclese. Agnew. Chem. 31, 1617-1619.
25. Erie, D., et al (1989) Melting behaviour of covalently closed single-stranded, circular DNA. Biochem. 28, 268-73.
26. Ashley, G., and Kushlan, D. (1991) Chemical synthesis of oligodeoxynucleotide dumbbells. Biochem 30, 2927-33.
27. Bradley, L M., et al (1992). Long-term CD4+ memory T cells from the spleen lack MEL-14, the lymph node homing receptor. J. Immunol. 148:324-331.
28. Hicke, B., et al (1996) DNA aptamers block L-selectin function in vivo. J. Clin. Invest. 98, 2688-2692.
29. Zucker, M. (1994) Prediction of RNA secondary structure by energy minimisation. Methods Mol. Biol. 25, 267-94.
30. Yang, Q. et al, DNA ligands that bind tightly and selectively to cellobiose, Proc. Natl. Acad. Sci. USA 95, 5462-7 (1998).

1. An aptamer comprising a circular oligonucleotide defining one to four target binding regions.

2. The aptamer of claim 1 which defines two, three or four target binding regions wherein said binding regions are separated by at least partially duplex regions.

3. The aptamer of claim 1 which defines one or more protein, cellular, cell component or material binding region.

4. The aptamer of claim 3 wherein the protein binding region is a thrombin binding region

5. The aptamer of claim 3 wherein the cellular binding region is an L-selectin binding domain.

6. The aptamer of claim 1 consisting of nucleotides.

7. The aptamer of claim 1 consisting of RNA.

8. The aptamer of claim 1 consisting of DNA.

9. An aptamer comprising an oligonucleotide defining two, three or four thrombin binding quadruplex regions separated by at least partially duplex regions, wherein the quadruplex regions comprise a GGTWGGXGGTTGG (SEQ ID NO:3) sequence wherein M represents A or T and X represents a sequence of two to five nucleotides and/or nucleotide analogues.

10. The aptamer of claim 9 ligated at its termini to form a circular oligonucleotide.

11. The aptamer of claim 10 wherein the termini have been chemically ligated.

12. The aptamer of claim 10 wherein the termini have been enzymatically ligated.

13. The aptamer of claim 9 consisting of nucleotides.

14. The aptamer of claim 9 consisting of RNA.

15. The aptamer of claim 9 consisting of DNA.

16. The aptamer of claim 15 wherein X represents a sequence selected from TGT, GCA and TGA.

17. An aptamer represented by formula I:
5N D₁NwQxD₁D₂yQzD₂N 3N Formula I
wherein
Q represents a sequence GGTWGGXGGTTGG (SEQ ID NO:3) where M represents A or T and X represents a sequence of two to five nucleotides and/or nucleotide analogues;
w, x, y and z are the same or different and represent a sequence of zero to ten nucleotides and/or nucleotide analogues;
D₁ and D₂ are the same or different and each represent a sequence of zero to twenty-five nucleotides and/or nucleotide analogues with the proviso that D₁ and D₂ together comprise at least two nucleotides or nucleotide analogues;
D₁N and D₂N are the same or different and each represent a sequence of zero to fifty nucleotides and/or nucleotide analogues, wherein at least two consecutive nucleotides or nucleotide analogues of D₁N and/or D₂N are complimentary to at least two consecutive nucleotides or nucleotide analogues of D₁ and/or D₂, so as to allow duplex formation between complimentary nucleotides or nucleotide analogues.

18. The aptamer of claim 17 wherein the 5N terminus is phosphorylated.

19. The aptamer of claim 17 wherein w, x, y and z are the same or different and each represent zero, one or two nucleotides and/or nucleotide analogues.

20. The aptamer of claim 17 wherein D₁ and D₂ in total represent two to twenty nucleotides and/or nucleotide analogues.

21. The aptamer of claim 20 wherein D₁ and D₂ in total represent four to twelve nucleotides and/or nucleotide analogues.

22. The aptamer of claim 17 wherein D₁N and D₂N in total represent two to twenty nucleotides and/or nucleotide analogues.

23. The aptamer of claim 22 wherein D₁N and D₂N in total represent four to twelve nucleotides and/or nucleotide analogues.

24. The aptamer of claim 17 ligated at its termini to form a circular sequence of nucleotides and/or nucleotide analogues.

25. The aptamer of claim 24 wherein the termini have been chemically ligated.

26. The aptamer of claim 24 wherein the termini have been enzymatically ligated.

27. The aptamer of claim 17 consisting of nucleotides.

28. The aptamer of claim 17 consisting of RNA.

29. The aptamer of claim 17 consisting of DNA.

30. The aptamer of claim 17 wherein X represents a sequence selected from TGT, GCA and TGA.

31. The aptamer of claim 17 wherein D₁ and D₁N are selected from the following respective pairs:
CAG and CTG;
CAGC and GCTG;
CATGC and GCATG;
CATCGC and GCGATG.

32. The aptamer of claim 17 wherein D₂ and D₂N are selected from the following respective pairs:
CAC and GTG;
GCAC and GTGC;
GCTAC and GTAGC;
GACTAC and GTAGTC.

33. Aptamers selected from those with the following sequences:

| | | |
|---|---|---|
| DH6-1 | 5′ p CTG GGT TGG TGA GGT | (SEQ ID NO: 4) |
| | TGG TCA GCA CGG TTG GTG AGG | |
| | TTG GTG TG 3′ | |
| DH8-1 | 5′ p GCT GTG GTT GGT GAG | (SEQ ID NO: 5) |
| | GTT GGC AGC GCA CTG GTT GGT | |
| | GAG GTT GGG TGC 3′ | |
| DH10-1 | 5′ p GCA TGT GGT TGG TGA | (SEQ ID NO: 6) |
| | GGT TGG CAT GCG CTA CTG GTT | |
| | GGT GAG GTT GGG TAG C 3′ | |
| DH12-1 | 5′ p GCG ATG TGG TTG GTG | (SEQ ID NO: 7) |
| | AGG TTG GCA TCG CGA CTA CTG | |
| | GTT GGT GAG GTT GGG TAG TC | |
| | 3′ | |
| TS1-1 | 5′ p GCT GTG GTT GGT GAG | (SEQ ID NO: 8) |
| | GTT GGC AGC AGC CAA GGT AAC | |
| | CAG TAC AAG GTG CTA AAC GTA | |
| | ATG GCT TCG GCT 3′ | |
| TS2-1 | 5′ p GCT GTG GTT GGT GAG | (SEQ ID NO: 17) |
| | GTT GGC AGC AGC TGG CGG TAC | |
| | GGG CCG TGC ACC CAC TTA CCT | |
| | GGG AAG TGA GCT 3′ | |
| TS3-1 | 5′ p GCT GTG GTT GGT GAG | (SEQ ID NO: 18) |
| | GTT GGC AGC AGC CAT TCA CCA | |
| | TGG CCC CTT CCT ACG TAT GTT | |
| | CTG CGG GTG GCT 3′ | |
| DH8-Br | 5′ GCT GTG GTT GGB GAG | (SEQ ID NO: 12) |
| | GBB GGC AGC GCA CBG GBB - | |
| | GGB GAG GBB GGG BGC 3′ | |

where B=5-bromo-2′-deoxyuridine, 5-iodo-2′-deoxyuridine or other photoactive nucleotide analogue

34. An antidote aptamer comprising at least ten nucleotides and/or nucleotide analogues complimentary to a sequence of at least ten nucleotides and/or nucleotide analogues from an aptamer according to claim 17.

35. An antisense oligonucleotide to an aptamer of claim 17.

36. An antidote aptamer according to claim 34 ligated at its termini to form a circular oligonucleotide.

37. The antidote aptamer or the antisense oligonucleotide of claim 36 wherein the termini have been chemically ligated.

38. The antidote aptamer or the antisense oligonucleotide of claim 37 wherein the termini have been enzymatically ligated.

39. An aptamer according to claim 34 having the following sequence:

| | | |
|---|---|---|
| ADH8-1 | 5′ pGCA CCC AAC CTC ACC AAC | (SEQ ID NO: 19) |
| | CAG TGC GCT GCC AAC CTC ACC | |
| | AAG CAC AGC 3′. | |

40. A method of treatment of thrombosis in a patient requiring such treatment which comprises administering to said patient an effective amount of an aptamer according to claim 1.

41. A method of preventing or reducing coagulation of blood or blood derived products which comprises contacting the blood or blood derived product with an effective amount of an aptamer according to claim 1.

42. Use of a compound according to claim 1 in preparation of a medicament for the treatment of thrombosis.

43. A method for capturing leukocytes from a physiological fluid comprising contacting the physiological fluid with an effective amount of an aptamer according to claim 1.

44. A composition comprising an aptamer according to claim 1 or its antisense antidote together with one or more pharmaceutically acceptable carriers or excipients.

45. A composition according to claim 41 in oral dosage form.

46. A method for counteracting the effect of an aptamer according to claim 1 comprising contacting the aptamer with a counteracting effective amount of an antidote aptamer thereof.

47. An antisense oligonucleotide according to claim 35 ligated at its termini to form a circular oligonucleotide.
